(19) United States
(12) Patent Application Publication
(10) Pub. No.: US 2025/0072815 A1
(43) Pub. Date: Mar. 6, 2025

(54) DEVICE AND METHOD FOR, GENERATING INFORMATION ON VERTIGO, COMPUTER-READABLE RECORDING MEDIUM, AND COMPUTER PROGRAM

(71) Applicants: Industry Academic Cooperation Foundation, Hallym University, Chuncheon-si (KR); NEUROEARS CO., LTD., Anyang-si (KR)

(72) Inventors: Sung Kwang Hong, Seongnam-Si (KR); Eun-Cheon Lim, Seoul (KR); Changje Cho, Anyang-Si (KR)

(73) Assignees: Industry Academic Cooperation Foundation, Hallym University, Chuncheon-si (KR); NEUROEARS CO., LTD., Anyang-si (KR)

(21) Appl. No.: 18/398,258

(22) Filed: Dec. 28, 2023

(30) Foreign Application Priority Data
Aug. 29, 2023 (KR) 10-2023-0113797

(51) Int. Cl. A61B 5/00; A61B 3/00; A61B 3/113; A61B 5/11; G06T 7/11

(52) U.S. Cl. CPC A61B 5/4023; A61B 3/0025; A61B 3/113; A61B 5/1128; G06T 7/11; G06T 2207/20081; G06T 2207/20084; G06T 2207/30041

(57) ABSTRACT

Provided are a device and method for generating information on vertigo by tracking changes in eyes and head position, a computer-readable recording medium, and a computer program. More specifically, according to the device and method, movement of a patient's eyes and head is tracked in a video of the patient on the basis of a deep learning model to generate information on vertigo. The computer program for implementing the method is stored in the computer-readable recording medium.

### CROSS-REFERENCE TO RELATED APPLICATION

This application claims priority to and the benefit of Korean Patent Application No. 10-2023-0113797 filed in the Korean Intellectual Property Office on Aug. 29, 2023, the disclosure of which is incorporated herein by reference in its entirety.

### BACKGROUND

### 1. Field of the Invention

The present invention relates to a technique for generating information on vertigo by tracking changes in eyes and head position, and more particularly, to a device and method for generating information on vertigo by tracking movement of a patient's eyes and head in a video of the patient on the basis of a deep learning model, a recording medium on which a program for implementing the method is stored, and the computer program stored in the recording medium.

### 2. Discussion of Related Art

The content of this section merely provides background information of embodiments described in this specification and does not necessarily constitute prior art.

Vertigo is a term collectively referring to all symptoms where a person feels like he or she or objects around him or her are moving even though they are still, and such vertigo mostly occurs due to dysfunction of the peripheral vestibular nervous system. When there is an abnormality in the peripheral vestibular nervous system, the vestibulo-ocular reflex, which is the ability to fix one's gaze in accordance with changes in head position, is lost, and thus the eyes are not fixed and involuntarily move, leading to vertigo. Movement of the eyes caused by an abnormality in the vestibular function is called nystagmus. When people suffer from vertigo, doctors determine whether their vestibular functions are normal by assessing nystagmus. Nystagmus is caused not only by abnormalities in the peripheral vestibular nervous system but also by abnormalities in the central nervous system. Doctors identify whether vertigo that patients suffer from is a problem in their peripheral vestibular system or their central nervous systems. In other words, nystagmus observation is an important test tool that provides the greatest diagnostic information in treating patients with vertigo. Nystagmus includes movement on three axes: 1) horizontal directions (left and right), 2) vertical directions (up and down), and 3) rotational directions (clockwise and counterclockwise). Currently, such nystagmus observation may be performed through Frenzel glasses, infrared photography, video Frenzel glasses, and video nystagmus.

However, Frenzel glasses or video Frenzel glasses which are devised to directly observe movement of the eyes require direct ocular observation for assessment and thus may vary in diagnostic accuracy depending on experience of a doctor who determines minute movement of the eyes. Videonystagmography equipment which outputs nystagmus as a graph to increase the diagnostic accuracy requires a rapid eye-tracking technology with high accuracy and thus is expensive, which hinders wide use of videonystagmography equipment.

In particular, in a head impulse test that is used to most appropriately determine the vestibulo-ocular reflex which is movement of the eyes in accordance with changes in head position, the patient's eye movement that occurs while quickly moving the patient's head left and right or up and down is observed with unaided eyes. Therefore, the test result may vary depending on the skill level of a person who performs the test. Also, a video head impulse test that is carried out to objectify the result is performed through equipment additionally including a gyro sensor for measuring the speed of head movement and an eye tracker for calculating the speed of eye movement. In other words, such equipment makes it possible to output a head impulse test result or assess a ratio of eye movement to head movement as a gain and determine whether the vestibular function is abnormal. Also, while eye movement is observed, noise may be generated due to blinking and the like. Since current equipment does not have a function of removing the noise, it is necessary for a tester to tell a patient to keep his or her eyes open for a certain time period, which is inconvenient. Since doctors frequently identify patients' states only through test results, the results may be distorted by such eye blinking.

### SUMMARY OF THE INVENTION

The present invention is directed to providing a device for generating information on vertigo in which an algorithm developed through a deep learning model automatically removes noise, such as eye blinking and the like, not only from eye movement videos captured by generally used videonystagmography equipment but also from eye movement and head movement videos captured by various types of equipment and tracks and quantifies only eye movement so that doctors may use the values in diagnosis.

The present invention is also directed to providing a device for generating information on vertigo that does not require a person to wear goggles equipped with complex tools, such as a gyro-sensor for tracking and calculating head movement and an eye tracker for tracking and calculating eye movement, for a video head impulse test which is used as an objective test, allows scenes of a head impulse test of rapidly turning a patient's head left and right or up and down to be captured as a video through a simple camera, such as a web camera, a smartphone, or the like, allows a learning algorithm to calculate the speed of eye movement and the speed of head movement only from the video, and thus allows any device for recording a video to perform an important test to distinguish between peripheral and central vertigo, which allows telemedicine in vertigo diagnosis.

The present invention is also directed to providing a recording medium in which a program for implementing a vertigo diagnosis method is stored and the computer program stored in the recording medium.

Objectives of this specification are not limited to those described above, and other objectives which have not been described will be clearly understood by those of ordinary skill in the art from the following description.

According to an aspect of the present invention, there is provided a device for generating information on vertigo, the device including an eye movement learning part configured to identify an eye portion in an eye video of a patient's eye, extract an eye image from each frame, recognize a pupil center in the eye image, calculate coordinates of the pupil center, and learn movement of the eye, an eye movement output part configured to receive information learned by the eye movement learning part and output information on eye movement from a video of the patient, a head movement output part configured to output information on head movement from the video of the patient, a computation part configured to receive the information on the eye movement and the information on the head movement from the eye movement output part and the head movement output part, calculate an eye movement speed and a head movement speed, and calculate a gain, and an information generating part configured to receive the calculated gain from the computation part and generate information on vertigo.

The eye movement learning part may include an object segmentation model generation module configured to segment the eye image extracted from each frame into sclera, iris, and pupil regions and generate an object segmentation model, an eyeblink identification module configured to receive information on an intermediate layer in the generation of the object segmentation model, learn an anatomical structure of a vicinity of the eye, identify eye blinking, and generate an eyeblink classification model, and a sight-tracking model generation module configured to receive the information on the intermediate layer in the generation of the object segmentation model and generate a model for a direction of sight.

The eye movement learning part may infer a position of the pupil covered by eyelid when the patient closes the eye on the basis of the eyeblink classification model and recognize the pupil center in the eye before the pupil is covered.

The eye movement learning part may further include a graph output module configured to output information on the pupil center on graph for three types of movements (horizontal, vertical, and rotational).

The eye movement output part may calculate coordinates of an upper end, a lower end, a left end, and a right end of a scleral edge of the patient's eye from the video of the patient, receive the coordinates of the pupil center calculated by the eye movement learning part, calculate a direction vector of the eye by converting two-dimensional (2D) coordinates representing a direction of the pupil center into three-dimensional (3D) coordinates using coordinates of an upper end, a lower end, a left end and a right end of a scleral edge of the patient's eye and the coordinates of the pupil center, and output the information on the eye movement.

The head movement output part may calculate coordinates of a nose of the patient, a left end and a right end of a scleral edge of the eye, and a forehead center, calculate a direction vector of the head by converting 2D coordinates of a head direction into 3D coordinates using coordinates of a nose of the patient, a left end and a right end of a scleral edge of the eye and a forehead center, and output the information on the head movement.

The computation part may calculate the gain as follows:

$$Gain = \frac{\vert eye peak index - head peak index - FPS \vert}{F P S} .$$

frames per second (FPS): a time resolution in the video of the patient
head peak index: a time point when the head is moved by a predetermined angle
eye peak index: a time point of moving, after the head moves, the pupil backward relative to the head within one second

The information generating part may generate information on whether a vestibular function is abnormal using the eye movement output from the eye movement output part and the gain.

According to another aspect of the present invention, there is provided a method of generating information on vertigo, the method including (a) identifying an eye portion in an eye video of a patient's eye, extracting an eye image from each frame, recognizing a pupil center in the eye image, calculating coordinates of the pupil center, and learning movement of the pupil center, (b) outputting information on eye movement from a video of a patient using information on eye movement learned in operation (a), (c) outputting information on head movement from the video of the patient, (d) calculating an eye movement speed and a head movement speed using the information on the eye movement and the information on the head movement output in operation (b) and operation (c) to calculate a gain, and (e) generating information on vertigo using the gain calculated in operation (d).

Operation (a) may include (a-1) segmenting the eye image extracted from each frame into sclera, iris, and pupil regions and generating an object segmentation model, (a-2) learning an anatomical structure of a vicinity of the eye using information on an intermediate layer of operation (a-1), identifying eye blinking, and generating an eyeblink classification model, and (a-3) generating a model for a direction of sight using the information on the intermediate layer of operation (a-1).

Operation (a) may include inferring a position of the pupil covered by eyelid when the patient closes the eye on the basis of the eyeblink classification model generated in operation (a-2) and recognizing the pupil center in the eye before the pupil is covered.

Operation (a) may further include outputting information on the pupil center on graph for three types of movements (horizontal, vertical and rotational).

Operation (b) may include calculating coordinates of an upper end, a lower end, a left end, and a right end of a scleral edge of the patient's eye from the video of the patient, calculating a direction vector of the eye by converting 2D coordinates representing a direction of the pupil center into 3D coordinates using coordinates of an upper end, a lower end, a left end and a right end of a scleral edge of the patient's eye and the coordinates of the pupil center calculated in operation (a), and outputting the information on the eye movement.

Operation (c) may include calculating coordinates of a nose of the patient, a left end and a right end of a scleral edge of the eye, and a forehead center, calculating a direction vector of the head by converting 2D coordinates of a head direction into 3D coordinates using coordinates of a nose of the patient, a left end and a right end of a scleral edge of the eye and a forehead center, and outputting the information on the head movement.

Operation (d) may include calculating the gain as follows:

$$Gain = \frac{\vert eye peak index - head peak index - FPS \vert}{F P S} .$$

frames per second (FPS): a time resolution in the video of the patient
head peak index: a time point when the head is moved by a predetermined angle
eye peak index: a time point of moving, after the head moves, the pupil backward relative to the head within one second

Operation (e) may include generating information on whether a vestibular function is abnormal using the eye movement output in operation (b) and the gain calculated in operation (d).

According to another aspect of the present invention, there is provided a computer-readable recording medium in which a program for implementing the method of generating information on vertigo is stored.

According to another aspect of the present invention, there is provided a computer program stored in a computer-readable recording medium to implement the method of generating information on vertigo.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram schematically illustrating a device for generating information on vertigo according to an exemplary embodiment of this specification;

FIG. 2 is a diagram schematically illustrating a process of learning eye movement according to this specification;

FIG. 3 is a diagram illustrating a process of generating an object segmentation model, recognizing a pupil center, and outputting movement of the pupil center in a graph;

FIG. 4 is a diagram illustrating generation of an artificial intelligence model for recognizing a pupil center;

FIG. 5 is a diagram illustrating a method of measuring a speed of eye change in accordance with head movement;

FIG. 6 is a flowchart illustrating a method of generating information on vertigo according to an exemplary embodiment of this specification; and

FIG. 7 is a flowchart illustrating a process in which an eye movement learning part recognizes a pupil center and calculates coordinates of the pupil center according to an exemplary embodiment of this specification.

### DETAILED DESCRIPTION OF EXEMPLARY EMBODIMENTS

Advantages and features of the present invention and methods of achieving them will become clear with reference to exemplary embodiments described below in detail in conjunction with the accompanying drawings. However, the present invention is not limited to the embodiments disclosed below and may be implemented in various different forms, the embodiments are provided only to fully convey the scope of the present invention to those of ordinary skill in the technical field to which the present invention pertains, and the present invention is only defined by the scope of the claims.

Terminology used herein is for describing the embodiments and is not intended to limit the present invention. In this specification, singular forms also include plural forms unless specifically stated otherwise. As used herein, “comprise (or include)” and/or “comprising (including)” may include components and processes in addition to stated components and processes. Throughout the specification, like reference numerals refer to like components. “And/or” includes each and all combinations of one or more mentioned items.

Unless otherwise defined, all terms (including technical and scientific terms) used in this specification may be used with a meaning commonly understood by those of ordinary skill in the art. Also, terms defined in commonly used dictionaries are not interpreted ideally or excessively unless explicitly specifically defined.

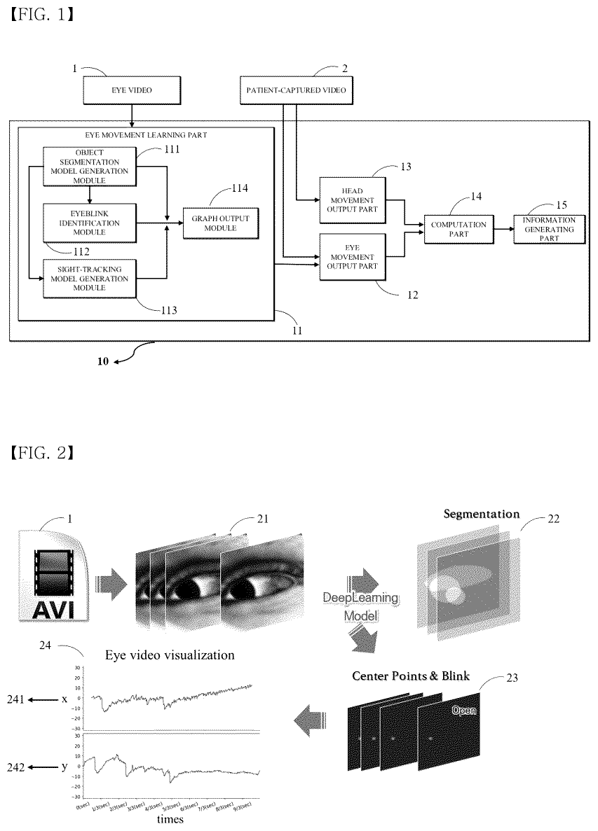

FIG. 1 is a block diagram schematically illustrating a device for generating information on vertigo according to an exemplary embodiment of this specification. Referring to FIG. 1, a device for generating information on vertigo 10 according to the exemplary embodiment may learn eye movement from an eye video 1 of a patient's eye. Eye movement in accordance with head movement may be measured on the basis of training information for movement of a pupil center, and the information on vertigo may be generated. In this way, the information on vertigo may be generated using the eye video 1 and a patient-captured video 2. The eye video 1 and the patient-captured video 2 may be videos recorded with a recording device for video recording. Video data has various formats such as MP4, MOV, WMV, AVI, MKV, and the like, and video data in accordance with this specification is not limited to a specific video file format.

The device for generating information on vertigo 10 may include an eye movement learning part 11, an eye movement output part 12, a head movement output part 13, a computation part 14, and an information generating part 15.

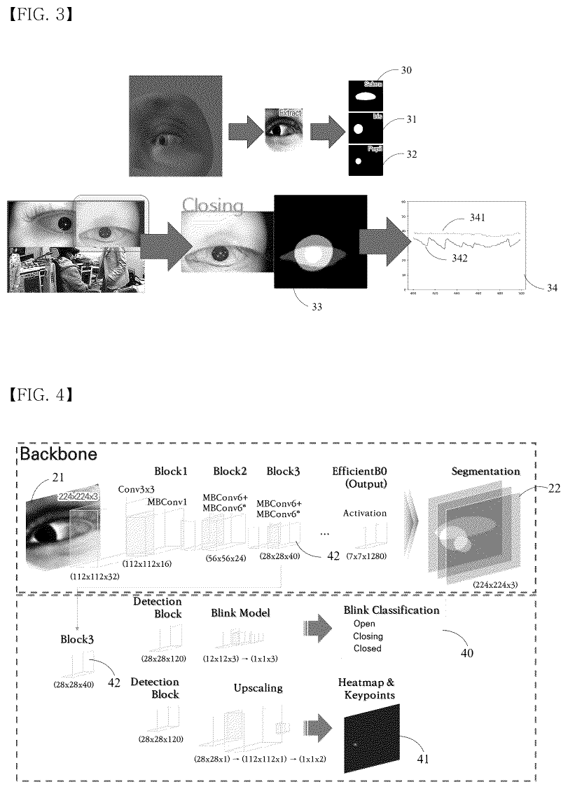

FIG. 2 is a diagram schematically illustrating a process of learning eye movement according to this specification.

Referring to FIGS. 1 and 2, the eye movement learning part 11 may identify an eye portion in the eye video 1 and extract an eye image 21 from each video frame. The eye movement learning part 11 may recognize a pupil center in the eye image 21, calculate coordinate values of the pupil center, and learn eye movement. Image data has various formats such as JPG, PNG, GIF, SVG, and the like, and image data in accordance with this specification is not limited to a specific image file format.

The eye movement learning part 11 may include an object segmentation model generation module 111, an eyeblink identification module 112, and a sight-tracking model generation module 113.

The object segmentation model generation module 111 may segment the eye in the eye image 21 into a sclera, an iris, and a pupil and generate an object segmentation model 22. The eye movement learning part 11 may use information of the object segmentation model 22 in recognizing the pupil center.

The object segmentation model generation module 111 can provide information on sclera, iris, and pupil regions, which is not provided by a nystagmus test method employing an infrared camera according to the related art.

The eyeblink identification module 112 may receive information on an intermediate layer in the generation of the object segmentation model 22, learn an anatomical structure of the vicinity of the eye, identify eye blinking, and generate an eyeblink classification model for classifying stages of eye blinking.

The eye movement learning part 11 may infer a position of the pupil covered by eyelid when the patient closes the eye on the basis of the eyeblink classification model and recognize the pupil center in the eye before the pupil is covered. Also, noise caused by eye blinking may be removed, and thus it is possible to prevent a result from being distorted by eye blinking.

The sight-tracking model generation module 113 may receive the information on the intermediate layer in the generation of the object segmentation model 22 and output a sight-tracking model which is a model for the direction of sight. The eye movement learning part 11 may use information of the sight-tracking model in recognizing the pupil center.

The eye movement learning part 11 may further include a graph output module 114. The graph output module 114 may output coordinate values of the pupil center calculated on the basis of information of the object segmentation model 22, the eyeblink classification model, and the sight-tracking model 23 on graph for three types of movements 24. The graph for three types of movements 24 may be output as a horizontal movement graph 241 showing left and right movements of the pupil center, a vertical movement graph 242 showing up and down movements, and a rotational movement graph (not shown) showing clockwise and counterclockwise movements.

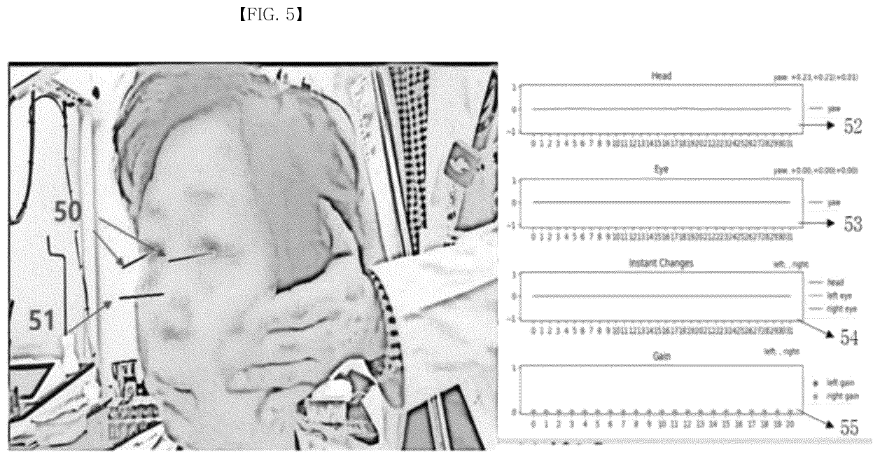

FIG. 3 is a diagram illustrating a process of generating an object segmentation model, recognizing a pupil center, and outputting movement of the pupil center in a graph.

Referring to FIGS. 1 and 3, the object segmentation model generation module 111 may generate the object segmentation model 22 from the patient's eye image extracted by the eye movement learning part 11. The object segmentation model 22 may segment the patient's eye image into a sclera 30, an iris 31, and a pupil 32. Also, the object segmentation model generation module 111 may output an object integration image 33 obtained by integrating images of the sclera 30, the iris 31, and the pupil 32 which have been segmented. The eye movement learning part 11 may track movement of the pupil center in real time using the object integration image 33.

The eye movement learning part 11 may output information on the pupil center which is recognized using the object integration image 33, on a pupil center movement graph 34 through the graph output module 114.

The pupil center movement graph 34 may be output on the basis of the information on the pupil center provided by a deep learning model. Also, the pupil center movement graph 34 may be calculated more precisely through the center of gravity of the pupil 32 which has been segmented. The pupil center movement graph 34 may be output as a horizontal movement graph 341, a vertical movement graph 342, and a rotational movement graph (not shown).

FIG. 4 is a diagram illustrating generation of an artificial intelligence model for recognizing a pupil center.

Referring to FIG. 4, to appropriately learn information on a pupil center, artificial intelligence may generate the object segmentation model 22, an eyeblink classification model 40, and a sight-tracking model 41 through a deep learning model.

The object segmentation model generation module 111 may generate the object segmentation model 22 from a convolutional neural network (CNN) deep learning model using the eye image 21. Since the CNN deep learning model is well-known technology to those of ordinary skill in the art, detailed description thereof will be omitted.

The eyeblink classification model 40 and the sight-tracking model 41 may be generated using information on an intermediate layer 42 in the generation process of the object segmentation model 22. When the information on the intermediate layer 42 is used, it is possible to learn information on the pupil center more efficiently than with a deep learning model according to the related art.

The eyeblink classification model 40 may classify eye-blinking motion as an open state in which the eye is open, a closing state in which the eye is being closed, or a closed state in which the eye is closed on the basis of the information on the intermediate layer 42.

The sight-tracking model 41 may represent a direction of sight with a heatmap and keypoints on the basis of the information on the intermediate layer 42.

According to this specification, the eye movement learning part 11 may learn the information on the pupil center using at least one of the object segmentation model 22, the eyeblink classification model 40, and the sight-tracking model 41.

As an example, the eye movement learning part 11 may learn eye movement only using information of the eyeblink classification model 40 and the sight-tracking model 41. In this case, information of the object segmentation model 22 is not used, and thus it is possible not to perform computation of the intermediate layer 42 which takes a long time. Accordingly, the information on the pupil center can be learned and output faster than that in a deep learning model according to the related art.

The CNN deep learning model for learning eye movement is exemplary, and the present invention is not limited to the corresponding learning method.

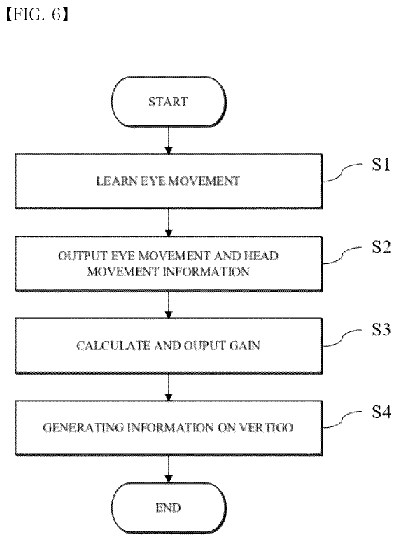

FIG. 5 is a diagram illustrating a method of measuring a speed of eye change in accordance with head movement.

Referring to FIGS. 1 and 5, the eye movement output part 12 and the head movement output part 13 may output information on eye movement and head movement of the patient from the patient-captured video 2.

The eye movement output part 12 may receive information on learned eye movement from the eye movement learning part 11. The eye movement output part 12 may output information on eye movement from the patient-captured video 2 on the basis of the information on the learned eye movement and output eye movement on a graph 53.

The eye movement output part 12 may calculate a direction vector of the eye to output information on eye movement. To calculate the direction vector of the eye, two-dimensional (2D) coordinates of at least four points may be converted into coordinates in a three-dimensional (3D) space using perspective-n-point (PnP) pose computation. Since PnP pose computation is well-known technology to those of ordinary skill in the art, detailed description thereof will be omitted.

To calculate a direction vector of the eye, the eye movement output part 12 may perform PnP pose computation on coordinates of an upper end, a lower end, a left end, and a right end of a scleral edge and 2D coordinates of the pupil center calculated by the eye movement learning part 11.

The eye movement output part 12 may output a pupil center marker 50 indicating the pupil center in accordance with eye movement of the patient in the patient-captured video 2.

The head movement output part 13 may output information on head movement from the patient-captured video 2 and output the head movement on a graph 52.

The head movement output part 13 may calculate a direction vector of the head to output the information on the head movement. The head movement output part 13 may perform PnP pose computation on coordinates of the nose, the left end and the right end of the scleral edge, and the forehead center in the patient-captured video 2 to calculate the direction vector of the head. Also, the head movement output part 13 may recognize head movement using movement of the nose center in the patient-captured video 2. In addition, the head movement output part 13 may output a nose center marker 51 indicating the patient's nose center in the patient-captured video 2.

In this specification, to output head movement, the coordinates of the nose, the left end and the right end of the scleral edge, and the forehead center in the patient-captured video 2 are used. However, the head movement is not necessarily output from the four sets of coordinates. As an example, coordinates of an upper end and a lower end of the scleral edge may be used instead of the coordinates of the left end and the right end of the scleral edge. Also, the head movement may be output from coordinates of any point present on the patient's face in the patient-captured video 2 in addition to the four sets of coordinates. Therefore, to output the head movement, it is not necessary to use the four sets of coordinates, and it is to be understood that the head movement may be output by performing PnP pose computation on coordinates of at least three points present on the patient's face in the patient-captured video 2.

The computation part 14 may calculate an eye movement speed and a head movement speed using the information on the eye movement and the head movement received from the eye movement output part 12 and the head movement output part 13. Then, the computation part 14 may calculate a gain representing a ratio of the eye movement speed to the head movement speed.

According to the related art, to calculate a gain, it is assumed that an object that a patient is looking at is at the center of a camera. Here, the gain may be obtained according to Equation 1.

$$Gain = \frac{eye movement speed}{head movement speed} \qquad [ Equation l ]$$

However, a desired time resolution is not obtained from a video, and a prediction of a monocular fixation vector is inaccurate.

According to this specification, eye movement caused by head movement occurs at the same time and in the same direction as the head movement, and thus an eye fixation vector can be predicted by averaging binocular changes. The accuracy of an eye direction can be increased using the eye fixation vector.

According to an exemplary embodiment of this specification, the gain may be calculated according to Equation 2.

$$Gain = \frac{\vert eye peak index - head peak index - FPS \vert}{F P S} \qquad [ Equation 2 ]$$

frames per second (FPS): a time resolution in the patient captured video 2
head peak index: a time point when the head is moved by a predetermined angle
eye peak index: a time point of moving, after the head moves, the pupil backward relative to the head within one second

According to an exemplary embodiment of this specification, the “eye peak index” may correspond to a value of a time point at which a third quartile or more is first shown in the case of extracting only speed values when the eye and the head move in opposite directions.

Also, the computation part 14 may output a graph 54 showing instant changes of the head and eye and a graph 55 of gain. Here, changes and gains of the left eye and the right eye may be separately output.

The information generating part 15 may generate information on the patient's vertigo using the gain calculated by the computation part 14.

The gain allows clinical judgment on whether vestibular nerves are abnormal. When a person without a problem in vestibular nerves moves his or her head, his or her eyes move as much as the head. For this reason, the gain has a value approximate to 1.

However, when there is a problem in the vestibular nerves, the eyes do not move as much as the head. For this reason, the gain has a value smaller than 1.

For example, a gain of the right eye calculated from the patient-captured video 2 may be 0.7, and a gain of the left eye may be 1.23. In this case, the gain of the right eye has a smaller value than 1, which may represent an inferior function of the right vestibular nerve.

Also, the information generating part 15 may generate information on whether a function of the central vestibular nerve is abnormal and whether a function of the peripheral vestibular nerve is abnormal using the gain and the direction vector of eye movement calculated by the eye movement output part 12.

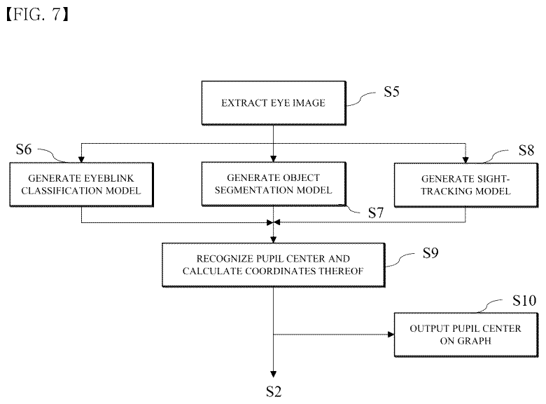

FIG. 6 is a flowchart illustrating a method of generating information on vertigo according to an exemplary embodiment of this specification.

Referring to FIGS. 1 and 6, eye movement may be learned from an eye video 1 obtained by capturing a patient's eye (S1). To learn eye movement, an eye portion may be identified in the eye video 1. Subsequently, an eye image may be extracted from each frame of the eye video 1. A pupil center may be recognized using the extracted eye image, and coordinates of the pupil center may be calculated so that eye movement may be learned.

FIG. 7 is a flowchart illustrating a process in which an eye movement learning part recognizes a pupil center and calculates coordinates of the pupil center according to an exemplary embodiment of the present invention.

Referring to FIGS. 1 and 7, eye images may be extracted from the eye video 1 (S5). An object segmentation model may be generated using the eye images (S7). Also, information on an intermediate layer in the generation of the object segmentation model may be input to generate an eyeblink classification model (S6) and a sight-tracking model (S8). When the eyeblink classification model determines that the patient closes his or her eyes in the eye video 1, a pupil center may be recognized using an eye before the eye was closed. Subsequently, at least one of the object segmentation model, the eyeblink classification model, and the sight-tracking model may be used to recognize the pupil center and calculate coordinates of the pupil center (S9).

The eye movement learning operation S1 may further include an operation S10 of outputting the coordinates of the pupil center on a graph. In this operation, the coordinates of the pupil center may be output on graph for three types of movements having horizontal directions, vertical directions, and rotational directions.

Subsequently, eye movement and head movement information of the patient may be output from a patient-captured video 2 (S2). The eye movement of the patient may be output on the basis of the information on the pupil center learned in the eye movement learning operation S1. Also, information on the eye movement and the head movement may be output on a graph.

To output the information on the eye movement from the patient-captured video 2, 2D coordinates representing a direction of the pupil center may be converted into 3D coordinates using coordinates of an upper end, a lower end, a left end, and a right end of a scleral edge of the patient's eye and the coordinates of the pupil center calculated in the eye movement learning operation (S1), and a direction vector of the eye may be calculated.

To output the information on the head movement from the patient-captured video 2, 2D coordinates representing a head direction may be converted into 3D coordinates using coordinates of the nose, the left end and the right end of the scleral edge of the eye, and the forehead center, and a direction vector of the head may be calculated.

Subsequently, a gain may be calculated on the basis of the eye movement and head movement of the patient and output (S3). To calculate the gain, an eye movement speed and a head movement speed may be calculated using the eye movement and head movement data.

The gain may be calculated using the eye movement speed and the head movement speed. When a time resolution in the patient-captured video 2 is “FPS,” a time point when the head is moved by a predetermined angle is a “head peak index,” and a time point of moving, after the head moves, the pupil backward relative to the head within one second is an “eye peak index,” the gain may be calculated according to Equation 2 above.

Also, instant changes of the head and eye and the gain may be output on graphs. In this process, changes and gains of the left eye and the right eye may be separately output.

Subsequently, the information on vertigo may be generated on the basis of the gain (S4). The gain which is close to 1 represents that a function of the vestibular nerve is normal, and the gain which is far from 1 represents that the function of vestibular nerve is abnormal. Also, whether a function of the central vestibular nerve is abnormal and whether a function of the peripheral vestibular nerve is abnormal may be determined using the gain and the eye movement.

The method of generating information on vertigo described above according to the exemplary embodiment of the present invention may be implemented in the form of a computer-executable recording medium (or a computer program), for example, a program module that is stored in a computer-readable medium and executed by a computer.

The computer-readable medium may be a computer storage medium (e.g., a memory, a hard disk drive, a magnetic/optical medium, a solid-state drive (SSD), or the like). The computer-readable medium may be any available medium that is accessible by a computer. For example, the computer-readable medium includes all of volatile and non-volatile media and detachable and non-detachable media.

In addition, all or a part of the method of generating information on vertigo according to the exemplary embodiment of the present invention may include computer-executable instructions. The computer program may include programmable machine code instructions which are processed by a processor, and may be implemented in a high-level programming language, an object-oriented programming language, an assembly language, a machine language, or the like.

As described above, with a device and method for generating information on vertigo according to exemplary embodiments of the present invention, the present algorithm can be applied to existing test equipment to automatically remove noise, such as eye blinking and the like, and output only a result related to eye movement. Also, eye movement and head movement are calculated from an eye video of a patient and a video of the patient through a deep learning model, and thus it is possible to test for vertigo simply through video capturing without complex equipment.

In addition, with a device and method for generating information on vertigo according to exemplary embodiments of the present invention, a head impulse test is possible on the basis of an eye video of a patient and a video of the patient. Accordingly, telemedicine is possible in vertigo diagnosis, which can contribute to healthcare.

Effects of the present invention are not limited to those described above, and other effects which have not been described will be clearly understood by those of ordinary skill in the art from the above description.

Although exemplary embodiments of the present invention have been described and illustrated using specific terms, the terms are only for clearly describing the present invention. It is obvious that various modifications and alterations can be made from the exemplary embodiments and described terms of the present invention without departing from the technical spirit and scope of the following claims. The modified embodiments should not be understood separately from the spirit and scope of the present invention and should be construed as falling within the scope of the claims of the present invention.

What is claimed is:

1. A device for generating information on vertigo, comprising:
an eye movement learning part configured to identify an eye portion in an eye video of a patient's eye, extract an eye image from each frame, recognize a pupil center in the eye image, calculate coordinates of the pupil center, and learn movement of the eye;
an eye movement output part configured to receive information learned by the eye movement learning part and output information on eye movement from a video of the patient;
a head movement output part configured to output information on head movement from the video of the patient;
a computation part configured to receive the information on the eye movement and the information on the head movement from the eye movement output part and the head movement output part, calculate an eye movement speed and a head movement speed, and calculate a gain; and
an information generating part configured to receive the calculated gain from the computation part and generate the information on vertigo.

2. The device of claim 1, wherein the eye movement learning part comprises:
an object segmentation model generation module configured to segment the eye image extracted from each frame into sclera, iris, and pupil regions and generate an object segmentation model;
an eyeblink identification module configured to receive information on an intermediate layer in the generation of the object segmentation model, learn an anatomical structure of a vicinity of the eye, identify eye blinking, and generate an eyeblink classification model; and
a sight-tracking model generation module configured to receive the information on the intermediate layer in the generation of the object segmentation model and generate a model for a direction of sight.

3. The device of claim 2, wherein the eye movement learning part infers a position of the pupil covered by eyelid when the patient closes the eye on the basis of the eyeblink classification model and recognizes the pupil center in the eye before the pupil is covered.

4. The device of claim 1, wherein the eye movement learning part further comprises a graph output module configured to output information on the pupil center on graph for three types of movements (horizontal, vertical, and rotational).

5. The device of claim 1, wherein the eye movement output part calculates coordinates of an upper end, a lower end, a left end, and a right end of a scleral edge of the patient's eye from the video of the patient, receives the coordinates of the pupil center calculated by the eye movement learning part, calculates a direction vector of the eye by converting two-dimensional (2D) coordinates representing a direction of the pupil center into three-dimensional (3D) coordinates using coordinates of an upper end, a lower end, a left end and a right end of a scleral edge of the patient's eye and the coordinates of the pupil center, and outputs the information on the eye movement.

6. The device of claim 1, wherein the head movement output part calculates coordinates of a nose of the patient, a left end and a right end of a scleral edge of the eye, and a forehead center, calculates a direction vector of the head by converting two-dimensional (2D) coordinates of a head direction into three-dimensional (3D) coordinates using coordinates of a nose of the patient, a left end and a right end of a scleral edge of the eye and a forehead center, and outputs the information on the head movement.

7. The device of claim 1, wherein the computation part calculates the gain as follows:
$$Gain = \frac{\vert eye peak index - head peak index - FPS \vert}{F P S} .$$
frames per second (FPS): a time resolution in the video of the patient
head peak index: a time point when the head is moved by a predetermined angle
eye peak index: a time point of moving, after the head moves, the pupil backward relative to the head within one second

8. The device of claim 1, wherein the information generating part generate information on whether a vestibular function is abnormal using the eye movement output from the eye movement output part and the gain.

9. A method of generating information on vertigo, comprising:
(a) identifying an eye portion in an eye video of a patient's eye, extracting an eye image from each frame, recognizing a pupil center in the eye image, calculating coordinates of the pupil center, and learning movement of the eye;
(b) outputting, by a processor, information on eye movement from a video of a patient using information on the eye movement learned in operation (a);
(c) outputting, by a processor, information on head movement from the video of the patient;
(d) calculating, by a processor, an eye movement speed and a head movement speed using the information on the eye movement and the information on the head movement output in operation (b) and operation (c) to calculate a gain; and
(e) generating, by a processor, information on vertigo using the gain calculated in operation (d).

10. The method of claim 9, wherein operation (a) comprises:
(a-1) segmenting, by a processor, the eye image extracted from each frame into sclera, iris, and pupil regions and generating an object segmentation model;
(a-2) learning, by a processor, an anatomical structure of a vicinity of the eye using information on an intermediate layer of operation (a-1), identifying eye blinking, and generating an eyeblink classification model; and
(a-3) generating, by a processor, a model for a direction of sight using the information on the intermediate layer of operation (a-1).

11. The method of claim 10, wherein operation (a) comprises inferring, by a processor, a position of the pupil covered by eyelid when the patient closes the eye on the basis of the eyeblink classification model generated in operation (a-2) and recognizing the pupil center in the eye before the pupil is covered.

12. The method of claim 9, wherein operation (a) further comprises outputting, by a processor, information on the pupil center on graph for three types of movements (horizontal, vertical and rotational).

13. The method of claim 9, wherein operation (b) comprises calculating, by a processor, coordinates of an upper end, a lower end, a left end, and a right end of a scleral edge of the patient's eye from the video of the patient, calculating a direction vector of the eye by converting two-dimensional (2D) coordinates representing a direction of the pupil center into three-dimensional (3D) coordinates using coordinates of an upper end, a lower end, a left end and a right end of a scleral edge of the patient's eye and the coordinates of the pupil center calculated in operation (a), and outputting the information on the eye movement.

14. The method of claim 9, wherein operation (c) comprises calculating, by a processor, coordinates of a nose of the patient, a left end and a right end of a scleral edge of the eye, and a forehead center, calculating a direction vector of the head by converting two-dimensional (2D) coordinates of a head direction into three-dimensional (3D) coordinates using coordinates of a nose of the patient, a left end and a right end of a scleral edge of the eye and a forehead center, and outputting the information on the head movement.

15. The method of claim 9, wherein operation (d) comprises calculating, by a processor, the gain as follows:
$$Gain = \frac{\vert eye peak index - head peak index - FPS \vert}{F P S} .$$
frames per second (FPS): a time resolution in the video of the patient
head peak index: a time point when the head is moved by a predetermined angle
eye peak index: a time point of moving, after the head moves, the pupil backward relative to the head within one second

16. The method of claim 10, wherein operation (e) comprises generating, by a processor, information on whether a vestibular function is abnormal using the eye movement output in operation (b) and the gain calculated in operation (d).

17. A computer-readable recording medium in which a program for implementing the method of claim 1 is stored.

18. A computer program stored in a computer-readable recording medium to implement the method of claim 1.
